(19) United States
(12) Patent Application Publication
(10) Pub. No.: US 2012/0195843 A1
(43) Pub. Date: Aug. 2, 2012

(54) Emulsified Antiperspirant Composition And Method For Making Same

(76) Inventors: David Frederick Swaile, Cincinnati, OH (US); Gary Paul Shrum, Maineville, OH (US)

(21) Appl. No.: 13/446,164

(22) Filed: Apr. 13, 2012

Related U.S. Application Data
(62) Division of application No. 12/140,853, filed on Jun. 17, 2008.
(60) Provisional application No. 60/936,099, filed on Jun. 18, 2007.

(51) Int. Cl. A61K 8/92; A61Q 15/00

(52) U.S. Cl. 424/65

(57) ABSTRACT

Antiperspirant compositions that include a continuous phase employing a water-immiscible liquid and a structurant, and a disperse phase employing a solution of antiperspirant active in water.

### CROSS REFERENCE TO RELATED APPLICATION

This application is a divisional of U.S. application Ser. No. 12/140,853, filed on Jun. 17, 2008, which is incorporated herein by reference, which claims the benefit under 35 USC 119(e) to U.S. Application No. 60/936,099, filed Jun. 18, 2007.

### FIELD OF THE INVENTION

The present invention is directed to antiperspirant compositions that include a continuous phase employing a water-immiscible liquid and a structurant, and a disperse phase employing a solution of antiperspirant active in water. The compositions are preferably in a solid or semi-solid stick form. Methods for making such antiperspirant compositions are also described.

### BACKGROUND OF THE INVENTION

The state of the art includes emulsion antiperspirant sticks. For example, U.S. Pat. No. 6,458,345 (the “'345 patent”) discloses an emulsion stick comprising a continuous oil phase and a disperse aqueous phase. The continuous oil phase comprises a volatile silicone oil, a non-volatile hydrophobic oil, and a wax structurant. The '345 patent describes blending the oils and wax structurant together and then heating the same within a range of 80° C. to 100° C. so that the wax can melt and disperse throughout the oil phase. The volatile silicone oils disclosed by the '345 patent however can have a flash point below this processing temperature. Thus, manufacturing the disclosed compositions within the disclosed heating range creates a potentially explosive scenario, which at commercial-scale operations could be extremely dangerous.

### SUMMARY OF THE INVENTION

The present invention is directed to emulsified antiperspirant compositions that employ selected ingredients that can be processed without the concern of fire or explosion and/or without the need for special equipment. The present invention is also directed to methods for making such antiperspirant compositions.

### DETAILED DESCRIPTION OF THE INVENTION

The present invention may be understood more readily by reference to the following detailed description of illustrative and preferred embodiments. It is to be understood that the scope of the claims is not limited to the specific ingredients, methods, conditions, devices, or parameters described herein, and that the terminology used herein is not intended to be limiting of the claimed invention. Also, as used in the specification, including the appended claims, the singular forms “a,” “an,” and “the” include the plural, and reference to a particular numerical value includes at least that particular value, unless the context clearly dictates otherwise. When a range of values is expressed, another embodiment includes from the one particular value and/or to the other particular value. Similarly, when values are expressed as approximations, by use of the antecedent basis “about,” it will be understood that the particular values form another embodiment. All ranges are inclusive and combinable.

All percentages and ratios used herein are by weight of the total composition, and all measurements made are at 25° C., unless otherwise designated.

The compositions/methods of the present invention can comprise, consist of, and consist essentially of the features and/or steps of the invention described herein, as well as any of the additional or optional ingredients, components, steps, or limitations described herein.

The term “ambient conditions” as used herein refers to surrounding conditions at about one atmosphere of pressure, about 50% relative humidity and about 25° C.

The term “water-immiscible” as used herein refers to materials or mixtures of materials with less than 1% water solubility at 25° C., and preferably less than 0.1% water solubility at 25° C. Most preferable are materials with less than 0.01% water solubility at 25° C.

The term “volatile” as used herein refers to those materials which have a measurable vapor pressure as measured at 25° C. and 1 atmosphere. The term “moderately volatile material,” as used herein, refers to those materials with a vapor pressure below about 2 mmHg at 25° C. The term “low volatile material,” as used herein, refers to those materials with a vapor pressure below about 0.5 mmHg at 25° C. The term “nonvolatile material,” as used herein, refers to those materials with a vapor pressure below about 0.002 mmHg at 25° C. Vapor pressures can be measured in a variety of manners and are often available in a variety of chemical data bases that would be known to one skilled in the art. One such database is available from the Research Institute for Fragrance Materials.

The antiperspirant compositions of the present invention comprise a continuous phase and a disperse aqueous phase. The continuous phase includes one or more water-immiscible liquids and a structurant. The disperse phase includes a solution of antiperspirant active in water.

### I. Continuous Phase

A. Water-Immiscible Liquid

A representative, non-limiting list of suitable water-immiscible liquids includes any material that is suitable for application to the human body. This can include any hydrocarbon, ester, ether, silicone or fluorocarbon emollient known in the art. Volatile silicones are one preferred class of water-immiscible liquids. While any volatile silicone emollient can be used, preferred materials will have a flash point above 80° C. Examples of suitable volatile silicones include, but are not limited to, cyclohexamethylsiloxane, hexyl methicone, capryl methicone and linear or branched polydimethyl siloxanes containing 4 to 6 silicone atoms. Volatile esters, such as, for example, diisopropyl adipate, can also be employed.

The water-immiscible liquids are preferably selected to have a flash point that is higher than the melting point of the structurant; for example, a flash point that is at least about 3° C. above the melting point of the structurant. Thus, if the structurant has a melting point of 77° C., then a preferred water-immiscible liquid has a flash point equal to or above about 80° C. When more than one water-immiscible liquid is employed in the antiperspirant composition, the individual materials may have a flash point above or below the melting point of the structurant. If water-immiscible liquids having a flash point below the melting point of the structurant are employed, it is preferred that the continuous phase overall has a flash point above the melting point of the structurant. In one preferred embodiment, all of the water-immiscible liquids in the antiperspirant composition have a flash point above the melting point of the structurant.

In some of the exemplary embodiments, the water-immiscible liquid has a flash point above about 70° C. In other exemplary embodiments, the water-immiscible liquid has a flash point above about 80° C.

It is to be understood that the continuous phase may contain hydrophilic materials, so long as the continuous phase overall is water-immiscible.

The water-immiscible liquids are preferably employed at concentration levels of greater than 5%, by weight of the complete composition.

B. Structurant

Suitable structurants include polyethylene waxes, ozokerite waxes, carnuba waxes, and mixtures thereof. Other suitable structurant materials include N-acyl amino acid amides and esters; for example, N-Lauroyl-L-glutamic acid di-n-butylamide. These materials are described in greater detail in U.S. Pat. No. 3,969,087. 12-hydroxystearic acid and esters and amines of the same represent another class of useful structurants for the antiperspirant compositions of the present invention.

Fiber-forming structurants may also be employed. These materials create a network of fibers or strands that extend throughout the continuous phase to gel the liquids therein. Such materials are generally non-polymeric, being monomers or dimmers that can have a molecular weight below about 10,000. Exemplary fiber-forming structurant materials have been reviewed by Terech and Weiss in “Low Molecular Mass Gelators of Organic Liquids and the Properties of their Gels” Chem. Rev 97, 3133-3159 [1997] and by Terech in Chapter 8, “Low-molecular Weight Organogelators” of the book “Specialist Surfactants” edited by I. D. Robb, Blackie Academic Professional, 1997.

Another suitable structurant is a partially or fully esterified cellobiose according the following formula:

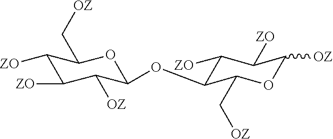

wherein each Z is independently hydrogen or an acyl group of the formula:

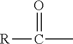

where R denotes a hydrocarbyl group containing from 4 to 22 carbon atoms. It one embodiment, not more than half of the Z groups are hydrogen.

Other suitable thickening or structuring agents for use in the present invention include, but are not limited to, fatty acid gellants, salts of fatty acids, hydroxy fatty acid gellants, esters and amides of fatty acid or hydroxy fatty acid gellants, cholesterolic materials, dibenzylidene alditols, lanolinolic materials, fatty alcohols, and triglycerides.

Suitable thickening or structuring agents can include, but are not limited to, solid salts of fatty acids wherein the fatty acid moiety has from about 12, from about 16 or from about 18 carbon atoms to about 40, to about 22, or about 20 carbon atoms. Suitable salt forming cations for use with these thickening or structuring agents include metal salts such as alkali metals (e.g. sodium and potassium), alkaline earth metals (e.g. magnesium), and aluminum. Preferred are sodium, potassium and aluminum salts. For example, suitable salt forming cations may be selected from the group consisting of sodium stearate, sodium palmitate, potassium stearate, potassium palmitate, sodium myristate, aluminum monostearate, and combinations thereof.

### II. Disperse Phase

The disperse phase generally includes water and an aqueous solution of an antiperspirant active. The antiperspirant active for use in the compositions of the present invention may include any compound, composition or other material having antiperspirant activity. By way of example only, the antiperspirant actives may include astringent metallic salts, especially inorganic and organic salts of aluminum, zirconium and zinc, as well as mixtures thereof. Particular antiperspirant active examples include, but are not limited to, aluminum-containing and/or zirconium-containing salts or materials, such as aluminum halides, aluminum chlorohydrate, aluminum hydroxyhalides, zirconyl oxyhalides, zirconyl hydroxyhalides, and mixtures thereof.

Aluminum salts useful in the present invention include those that conform to the formula:

Al₂(OH)ₐCl_{b}. xH₂O

wherein a is from about 0 to about 5; the sum of a and b is about 6; x is from about 1 to about 8; where a, b, and x may have non-integer values. For example, aluminum chlorohydroxides referred to as “3/4 basic chlorohydroxide,” wherein a is about 4.5; “5/6 basic chlorohydroxide,” wherein a=5; and “2/3 basic chlorohydroxide,” wherein a=4 may be used. Processes for preparing aluminum salts are disclosed in U.S. Pat. No. 3,887,692, issued to Gilman on Jun. 3, 1975; U.S. Pat. No. 3,904,741, issued to Jones et al. on Sep. 9, 1975; and U.S. Pat. No. 4,359,456 issued to Gosling et al. on Nov. 16, 1982. A general description of these aluminum salts can also be found in “Antiperspirants and Deodorants, Cosmetic Science and Technology Series” Vol. 20, 2nd edition, edited by Karl Laden. Mixtures of aluminum salts are described in British Patent Specification No. 1,347,950, filed in the name of Shin et al. and published Feb. 24, 1974. Zirconium salts for use in the present invention include those which conform to the formula:

ZrO(OH)₂₋ₐClₐ.xH₂O

wherein a is from about 0.5 to about 2; x is from about 1 to about 7; where a and x may both have non-integer values. These zirconium salts are described in Belgian Patent No. 825,146, issued to Schmitz on Aug. 4, 1975. Useful to the present invention are zirconium salt complexes that additionally contain aluminum and glycine, commonly known as “ZAG complexes”. These complexes contain aluminum chlorohydroxide and zirconyl hydroxy chloride conforming to the above-described formulas. Such ZAG complexes are described in U.S. Pat. No. 4,331,609, issued to On on May 25, 1982 and U.S. Pat. No. 4,120,948, issued to Shelton on Oct. 17, 1978.

Compositions of the present invention may additionally or alternatively employ a deodorant active; alternatively meaning that a deodorant active is substituted for an antiperspirant active. Suitable deodorant actives may be selected from the group consisting of antimicrobial agents (e.g., bacteriocides, fungicides), malodor-absorbing material, and combinations thereof. For example, antimicrobial agents may comprise cetyl-trimethylammonium bromide, cetyl pyridinium chloride, benzethonium chloride, diisobutyl phenoxy ethoxy ethyl dimethyl benzyl ammonium chloride, sodium N-lauryl sarcosine, sodium N-palmethyl sarcosine, lauroyl sarcosine, N-myristoyl glycine, potassium N-lauryl sarcosine, trimethyl ammonium chloride, sodium aluminum chlorohydroxy lactate, triethyl citrate, tricetylmethyl ammonium chloride, 2,4,4′-trichloro-2′-hydroxy diphenyl ether (triclosan), 3,4,4′-trichlorocarbanilide (triclocarban), diaminoalkyl amides such as L-lysine hexadecyl amide, heavy metal salts of citrate, salicylate, and piroctose, especially zinc salts, and acids thereof, heavy metal salts of pyrithione, especially zinc pyrithione, zinc phenolsulfate, farnesol, and combinations thereof.

The disperse phase may optionally contain other polar materials. A representative, non-limiting list of optional polar materials includes C1 to C20 monohydric alcohols; C2 to C40 dihydric or polyhydric alcohols; alkyl ethers of all such alcohols, e.g., C1-C4 alkyl ethers; polyalkoxylated glycols, e.g., propylene glycols and polyethylene glycols having from 2 to 30 repeating alkoxylate (e.g., ethoxylate or propoxylate) groups and polyglycerols having from 2 to 16 repeating glycerol moieties; and mixtures thereof. More particular exemplary polar materials include propylene glycol, hexylene glycol, dipropylene glycol, tripropylene glycol, glycerin, propylene glycol methyl ether, dipropylene glycol methyl ether, ethanol, n-propanol, n-butanol, t-butanol, 2-methoxyethanol, 2-ethoxyethanol, ethylene glycol, isopropanol, isobutanol, 1,4-butylene glycol, 2,3-butylene glycol, trimethylene glycol, 1,3-butanediol, 1,4,-butanediol, propylene glycol monoisostearate, PPG-3 myristyl ether, PEG-4 (also known as PEG-200), PEG-8 (also known as PEG-400), 1,2, pentanediol, PPG-14 butylether, dimethyl isosorbide, 1,2 hexanediol and combinations thereof. It is to be understood that polar materials other than those listed above may also be employed in the antiperspirant compositions described herein.

### III. Surfactants

Emulsifying surfactants are employed in the antiperspirant compositions to facilitate the formation of a stable emulsion containing the above-described continuous phase and disperse phase. The emulsifying surfactants may be anionic, cationic, zwitterionic and/or nonionic surfactants. Nonionic surfactants are preferred in the current invention. The proportion of emulsifier in the composition is often selected in the range up to 10% by weight and in many instances from 0.1 or 0.25 up to 5% by weight of the composition. Most preferred is an amount from 0.1 or 0.25 up to 3% by weight. Emulsifiers are frequently classified by HLB value. It is desirable, although not required, to use an emulsifier or a mixture of emulsifiers with an overall HLB value in a range from 2 to 10 preferably from 3 to 8.

It may be convenient to use a combination of two or more emulsifiers which have different HLB values above and below the desired value. By employing the two emulsifiers together in appropriate ratio, it is readily feasible to attain a weighted average HLB value that promotes the formation of an emulsion.

Many suitable emulsifiers of high HLB are nonionic ester or ether emulsifiers comprising a polyoxyalkylene moiety, especially a polyoxyethylene moiety, often containing from about 2 to 80, and especially 5 to 60 oxyethylene units, and/or contain a polyhydroxy compound such as glycerol or sorbitol or other alditol as hydrophilic moiety. The hydrophilic moiety can contain polyoxypropylene. The emulsifiers additionally contain a hydrophobic alkyl, alkenyl or aralkyl moiety, normally containing from about 8 to 50 carbons and particularly from 10 to 30 carbons. The hydrophobic moiety can be either linear or branched and is often saturated, though it can be unsaturated, and is optionally fluorinated. The hydrophobic moiety can comprise a mixture of chain lengths, for example those deriving from tallow, lard, palm oil, sunflower seed oil or soya bean oil. Such nonionic surfactants can also be derived from a polyhydroxy compound such as glycerol or sorbitol or other alditols. Examples of emulsifiers include ceteareth-10 to −25, ceteth-10-25, steareth-10-25 (i.e. C16 to C18 alcohols ethoxylated with 10 to 25 ethylene oxide residues) and PEG-15-25 stearate or distearate. Other suitable examples include C10-C20 fatty acid mono, di or tri-glycerides. Further examples include C18-C22 fatty alcohol ethers of polyethylene oxides (8 to 12 EO).

Examples of emulsifiers, which typically have a low HLB value, often a value from 2 to 6 are fatty acid mono or possibly diesters of polyhydric alcohols such as glycerol, sorbitol, erythritol or trimethylolpropane. The fatty acyl moiety is often from C14 to C22 and is saturated in many instances, including cetyl, stearyl, arachidyl and behenyl. Examples include monoglycerides of palmitic or stearic acid, sorbitol mono or diesters of myristic, palmitic or stearic acid, and trimethylolpropane monoesters of stearic acid.

A particularly desirable class of emulsifiers comprises dimethicone copolymers, namely polyoxyalkylene modified dimethylpolysiloxanes. The polyoxyalkylene group is often a polyoxyethylene (POE) or polyoxypropylene (POP) or a copolymer of POE and POP. The copolymers also include C1 to C12 alkyl groups as functional groups. Examples of suitable surfactants include DC5225 and DC 5200 (from Dow Corning), Abil EM 90 and EM 97 (from Gold Schmidt) and KF 6026, KF 6028, KF 6038 (from Shinetsu Silicones). The skilled artisan should appreciate that other emulsifying surfactants than those described above may also be used in antiperspirant compositions described herein.

### IV. Formation of the Emulsion

The continuous phase, disperse phase, and emulsifying surfactant are combined and then mixed or otherwise agitated sufficiently to form an emulsion. Typically, the disperse phase is added slowing to the continuous phase while the continuous phase is being vigorously agitated with a mixing system. The skilled artisan should appreciate the degree of mixing needed based on the desired phase ratio of the emulsion, its resulting viscosity and the desired batch size. The resulting emulsion can be further processed to create a consistent droplet size within the emulsion; for example, the emulsion may be processed by a mill to reduce droplet size and/or improve droplet size uniformity. Preferably, the emulsion is processed so that the entire batch experiences an equivalent amount of shear. A single-phase inline mill is one preferred apparatus for the additional, optional processing.

### V. Optional Ingredients

Antiperspirant compositions of the present invention may include one or more fragrance/perfume materials. In one preferred embodiment, the composition includes a fragrance material comprising a plurality of different perfume raw materials. Typical perfume levels in the present invention are 0.25 to 5%. Nonlimiting examples of fragrance materials include any known fragrances in the art or any otherwise effective fragrance materials. Typical fragrances are described in Arctander, “Perfume and Flavour Chemicals (Aroma Chemicals)”, Vol. I and II (1969) and Arctander, “Perfume and Flavour Materials of Natural Origin” (1960). U.S. Pat. No. 4,322,308, issued to Hooper et al., Mar. 30, 1982 and U.S. Pat. No. 4,304,679, issued to Hooper et al., Dec. 8, 1981 disclose suitable fragrance materials including, but not limited to, volatile phenolic substances (such as iso-amyl salicylate, benzyl salicylate, and thyme oil red), essence oils (such as geranium oil, patchouli oil, and petitgrain oil), citrus oils, extracts and resins (such as benzoin siam resinoid and opoponax resinoid), “synthetic” oils (such as Bergamot™ 37 and Bergamot™ 430, Geranium™ 76 and Pomeransol™ 314), aldehydes and ketones (such as B-methyl naphthyl ketone, p-t-butyl-A-methyl hydrocinnamic aldehyde and p-t-amyl cyclohexanone), polycyclic compounds (such as coumarin and beta-naphthyl methyl ether), esters (such as diethyl phthalate, phenylethyl phenylacetate, non-anolide 1:4).

Suitable fragrance materials may also include esters and essential oils derived from floral materials and fruits, citrus oils, absolutes, aldehydes, resinoides, musk and other animal notes (e.g., natural isolates of civet, castoreum and musk), balsamic, and alcohols (such as dimyrcetol, phenylethyl alcohol and tetrahydromuguol). For example, the antiperspirant compositions may comprise fragrances selected from the group consisting of decyl aldehyde, undecyl aldehyde, undecylenic aldehyde, lauric aldehyde, amyl cinnamic aldehyde, ethyl methyl phenyl glycidate, methyl nonyl acetaldehyde, myristic aldehyde, nonalactone, nonyl aldehyde, octyl aldehyde, undecalactone, hexyl cinnamic aldehyde, benzaldehyde, vanillin, heliotropine, camphor, para-hydroxy phenolbutanone, 6-acetyl 1,1,3,4,4,6 hexamethyl tetrahydronaphthalene, alpha-methyl ionone, gamma-methyl ionone, amyl-cyclohexanone, and mixtures thereof. Fragrance materials other than those listed above may also be employed.

The antiperspirant compositions can also include residue-masking agents to reduce the appearance of white residue arising from the antiperspirant active and structurant employed in the product. These masking agents can be incorporated into either the continuous or disperse phased depending on their water solublity. Exemplary residue-masking agents include isostearyl isostearate, glycereth-7-benzoate, C12-C15 alkyl benzoate, octyldodecyl benzoate, isostearyl lactate, isostearyl palmitate, benzyl laurate, laureth 4, laureth 7, oleth 2, PEG 4, PEG 12, isopropyl myristate isopropyl palmate, butyl stearate, polyethylene glycol methyl ethers, PPG 2 ceteareth 9, PPG 2 isodeceth 12, PPG 5 butyl ether, PPG 14 butyl ether, PPG 15 butyl ether, PPG 53 butyl ether, octyldodecanol, polydecene, mineral oil, petrolatum, phenyltrimethicone, dimethicone copolyol, and mixtures thereof. One preferred concentration level of the optional residue-masking agent is from about 3% to about 10%, by weight of the composition. But other concentration levels may also be used.

Antiperspirant compositions of the present invention may employ one or more additional ingredients. Nonlimiting examples of such optional ingredients include, but are not limited to, pH buffering agents, additional malodor controlling agents, emollients, humectants, soothing agents, dyes and pigments, medicaments, baking soda and related materials, preservatives, and soothing agents such as aloe vera, allantoin, D-panthenol, pantothenic acid derivatives (e.g., those disclosed in U.S. Pat. No. 6,495,149), avocado oil and other vegetative oils, and lichen extract.

### VI. Methods for Manufacturing Antiperspirant Compositions

Methods for manufacturing antiperspirant compositions are also provided herein. The description and appended claims include a listing of steps with either letter or numerical designations associated with the individual steps. It is to be understood that although they may, the methods and steps do not necessarily need to be performed in the order of listing or in accordance with their associated designations; for example, a step (d) may be performed before or after a step (b). Furthermore, although steps are listed individually, some steps may be performed simultaneously with other steps. Alternatively, the steps are all performed sequentially. Timing of the steps can vary. Also, there may or may not be delays between steps. And the methods described herein may include other steps than those explicitly listed and/or recited in the appended claims.

One exemplary method includes the steps of: (a) preparing an emulsion comprising a continuous oil phase and a disperse aqueous phase, wherein the continuous oil phase comprises one or more volatile liquids, wherein the continuous phase has a flash point above about 80° C., and wherein the aqueous phase comprises a solution of antiperspirant active in water; (b) providing a structurant; (c) heating the emulsion to a temperature from about 5° C. lower than the melting point of the structurant to about 25° C. higher than the melting point of the structurant; (d) combining the heated emulsion and the structurants (which may or may not be in a molten state before addition) to form an antiperspirant composition; and (e) cooling the antiperspirant composition, and/or allowing the antiperspirant composition to cool, to form a solid antiperspirant product.

A second exemplary method includes the steps of: (a) preparing an emulsion comprising a continuous oil phase and a disperse aqueous phase, the continuous oil phase comprising one or more volatile liquids, and the aqueous phase comprising a solution of antiperspirant active in water; (b) providing a structurant; (c) heating the emulsion to a temperature that is above the melting point of the structurant and below the lowest flash point of the one or more volatile liquids; (d) combining the heated emulsion and the structurant to form an antiperspirant composition; and (e) cooling the antiperspirant composition, and/or allowing the antiperspirant composition to cool, to form a solid antiperspirant product.

A third exemplary method includes the steps of: (a) combining one or more water-immiscible liquids with a structurant to form a water-immiscible liquid and structurant mixture; (b) providing an aqueous solution comprising an antiperspirant active; (c) preparing an emulsion comprising a continuous phase including the water-immiscible liquid and structurant mixture and a disperse phase including the aqueous solution to form an emulsified antiperspirant composition; (d) heating the water-immiscible liquid and structurant mixture, the aqueous solution, and/or the emulsified antiperspirant composition to a temperature above the melting point of the structurant and below the lowest flash point of the one or more water-immiscible liquids; and (e) cooling the emulsified antiperspirant composition, and/or allowing the emulsified antiperspirant composition to cool, to form a solid antiperspirant product.

### VII. Method of Use

The antiperspirant compositions provided herein may be topically applied to the axilla or other area of the skin in any known or otherwise effective method for controlling wetness and/or malodor associated with perspiration. Exemplary application levels include, for example, from about 0.1 gram per axilla to about 2.0 gram per axilla. The compositions are preferably applied to the axilla or other area of the skin one or more times daily, preferably once daily. Antiperspirant products according to the present invention can be applied prior to going to sleep or before a resting period—such application may increase the wetness protection efficacy as compared to applying the products prior to an active period.

### VIII. Examples

The following examples further describe and demonstrate embodiments within the scope of the present invention. The examples are given solely for the purpose of illustration and are not to be construed as limitations of the present invention as many variations thereof are possible without departing from the spirit and scope of the invention.

| | Example | Example | Example | Example | Example |
|---|---|---|---|---|---|
| Ingredient | A | B | C | D | E |
| Part I: Partial | | | | | |
| Continuous Phase | | | | | |
| Cyclopentasiloxane ¹ | 22.65 | | 21.65 | 20.45 | 17.65 |
| DC5200 ² | 1.20 | 1.20 | 1.20 | 1.20 | 1.20 |
| Fragrance | 1.35 | 1.75 | 1.35 | 1.35 | 1.35 |
| Hexyl Methicone ³ | | 22.25 | | | 5.00 |
| Mineral oil | | | | | |
| Part II: Disperse | | | | | |
| Phase | | | | | |
| ACH (50% solution) | 40.00 | 40.00 | 40.00 | | 40.00 |
| ZAG (30% solution) | | | | 55.00 | |
| propylene glycol | 5.00 | 5.00 | 5.00 | 5.00 | 5.00 |
| water | 12.30 | 12.30 | 12.30 | | 12.30 |
| Part III: Structurant | | | | | |
| Plus Remainder of | | | | | |
| Continuous Phase | | | | | |
| FinSolve TN | 6.50 | 6.50 | 6.50 | 6.00 | 6.50 |
| Ozocrite Wax | | | 12 | | |
| Performalene PL ⁴ | 11.00 | 11.00 | | 11.00 | 11.00 |

¹ DC 246 fluid from Dow Corning
² from Dow Corning
³ 41M10 from Cognis
⁴ from New Phase Technologies

All of these examples can be made via the following general process, which one skilled in the art will be able to alter to incorporate available equipment. The ingredients of Part I and Part II are mixed in separate suitable containers. Part II is then added slowly to Part I under agitation to assure the making of a water-in-silicone emulsion. The emulsion is then milled with suitable mill, for example a Greeco 1L03 from Greeco Corp, to create a homogenous emulsion. Part III is mixed and heated to 88° C. until the all solids are completely melted. The emulsion is then also heated to 88° C., and Part 3 ingredients are slowly added to the emulsion. The final mixture is then poured into an appropriate container, and allowed to solidify and cool to ambient temperature.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method for making an antiperspirant product, comprising the steps of:
(a) preparing an emulsion comprising a continuous oil phase and a disperse aqueous phase, wherein the continuous oil phase comprises one or more volatile liquids, wherein the continuous phase has a flash point above about 80° C., and wherein the aqueous phase comprises a solution of antiperspirant active in water;
(b) providing a structurant;
(c) heating the emulsion to a temperature from about 5° C. lower than the melting point of the structurant to about 10° C. higher than the melting point of the structurant;
(d) combining the heated emulsion and the structurant to form an antiperspirant composition; and
(e) cooling the antiperspirant composition, and/or allowing the antiperspirant composition to cool, to form a solid antiperspirant product.

2. The method of claim 1, wherein the temperature in step c) is from about 70° C. to about 90° C.

3. A method for making an antiperspirant product, comprising the steps of:
(a) preparing an emulsion comprising a continuous oil phase and a disperse aqueous phase, the continuous oil phase comprising one or more volatile liquids, and the aqueous phase comprising a solution of antiperspirant active in water;
(b) providing a structurant;
(c) heating the emulsion to a temperature that is above the melting point of the structurant and below the lowest flash point of the one or more volatile liquids;
(d) combining the heated emulsion and the structurant to form an antiperspirant composition; and
(e) cooling the antiperspirant composition, and/or allowing the antiperspirant composition to cool, to form a solid antiperspirant product.

4. The method of claim 3, wherein the volatile liquid is a silicone having a flash point above about 80° C.

5. The method of claim 3, wherein the structurant is selected from the group consisting of a polyethylene wax, an ozokerite wax, a carnuba wax, and mixtures thereof.

6. The method of claim 3, wherein the structurant comprises a fiber-forming structurant material.

7. The method of claim 3, wherein the structurant comprises a cellobiose compound.

8. A method for making an antiperspirant product, comprising the steps of:
(a) combining one or more water-immiscible liquids with a structurant to form a water-immiscible liquid and structurant mixture;
(b) providing an aqueous solution comprising an antiperspirant active;
(c) preparing an emulsion comprising a continuous phase including the water-immiscible liquid and structurant mixture and a disperse phase including the aqueous solution to form an emulsified antiperspirant composition;
(d) heating the water-immiscible liquid and structurant mixture, the aqueous solution, and/or the emulsified antiperspirant composition to a temperature above the melting point of the structurant and below the lowest flash point of the one or more water-immiscible liquids; and
(e) cooling the emulsified antiperspirant composition, and/or allowing the emulsified antiperspirant composition to cool, to form a solid antiperspirant product.

9. The method of claim 8, wherein the water-immiscible liquid has a flash point above about 80° C.

10. The method of claim 8, wherein the water-immiscible liquid is a volatile silicone.

11. The method of claim 8, wherein the structurant is selected from the group consisting of a polyethylene wax, an ozokerite wax, a carnuba wax, and mixtures thereof.

12. The method of claim 8, wherein the structurant comprises a fiber-forming structurant material.

13. The method of claim 8, wherein the structurant comprises a cellobiose compound.
